(19) United States
(12) Patent Application Publication
(10) Pub. No.: US 2003/0109714 A1
(43) Pub. Date: Jun. 12, 2003

(54) Transition metal mediated process

(76) Inventors: Neil Wishart, Holden, MA (US); Alena Rudolph, Worcester, MA (US); Kurt Ritter, Frankfurt (DE)

Correspondence Address: Gayle B. O'Brien, Abbott Bioresearch Center, 100 Research Drive, Worcester, MA, 01605-4314, US

(21) Appl. No.: 10/251,562

(22) Filed: Sep. 20, 2002

Related U.S. Application Data
(63) Continuation-in-part of application No. 10/104,165, filed on Mar. 22, 2002.
(60) Provisional application No. 60/278,072, filed on Mar. 22, 2001.

(51) Int. Cl. C07D 417/02; C07D 413/02; C 07D 4 3/02

(52) U.S. Cl. 548/222; 544/60; 544/137; 544/139; 544/369; 544/370; 546/198; 546/199; 548/304.7; 548/307.4

(57) ABSTRACT

This invention relates to a transition metal mediated process for the preparation of optionally substituted 2-amino-benzoxazoles and or 2-amino-benzimidazoles, which are useful as therapeutic agents or as intermediates in the synthesis of therapeutic agents.

### CROSS-REFERENCE TO RELATED APPLICATION

This application is a continuation-in-part of U.S. Ser. No. 10/104,165 filed Mar. 22, 2002, which claims priority to U.S. provisional application No. 60/278,072, filed Mar. 22, 2001, the entire teachings of the above applications are incorporated herein by reference.

### BACKGROUND OF THE INVENTION

Substituted or unsubstituted 2-amino-benzoxazoles and 2-amino-benzimidazoles are present in certain commercial compounds, such as therapeutic drugs. These compounds are known to have biological activity against a number of biological targets, for example, and not exclusively including, inhibitors or modulators of histamine receptors (for example see: Yanni et al., WO 9413299-A1), tyrosine kinase inhibitors (for an example see: Cheung et al, WO 0244156), bombesin antagonists (for an example see: Higginbottom et al., WO 0240475), dopamine antagonists (e.g. He et al., U.S. Pat. No. 6,284,759), rotamase (Wythes et al., WO 2000005231-A1), type 2 helper T cell function (JP 10330369-A), inosine-5′-monophosphate dehydrogenase (Saunders et al, WO 9840381-A1), G-protein coupled receptors (Sato et al, EP 806419-A1 and Biol. Pharm. Bull., 20(7), 752-755 (1997)), fibrinogen (Casanova et al., Diabetes, 46, Suppl. 1, 116A (1997)), peroxisome proliferator activated receptors (Smith, WO 9725042-A1), calpain (JP 08183771-A), HIV reverse transcriptase (Hoffman et al., U.S. Pat. No. 5,308,854-A), leukotriene function (Farina et al., J. Pharmacol. Exp. Ther., 271 (3), 1418-1426, (1994), Pal et al., EP 657451-A2), and integrins (Clark et al., WO 200049005-A1, 200050380-A1, 200061580-A1, 200068213-A1 and Brittain et al., WO 200005223-A2).

The preparation of substituted 2-amino-benzoxazoles or 2-amino-benzimidazoles has been achieved via a number of synthetic strategies. These include cyclodesulfurization of a substituted N-(2-hydroxyphenyl)- or a N-(2-aminophenyl)thiourea in the presence of either mercuric oxide (for example, Garin et al., J. Heterocyclic Chem., 27 (2), 221 (1990), and Perkins et al., Tet. Lett., 40 (6), 1103-1106, (1999)), nickel dioxide (e.g. Ogura et al, Chem. Pharm. Bull., 29(6), 1518 (1981)), potassium superoxide (e.g. Sung et al., Chem. Lett., (8), 1291-1294 (1986)), N,N′-dicyclohexylcarbodiimide (e.g. DE 3006671, Saunders et al, WO 9840381-A1) sodium hypochlorite and a phase transfer catalyst (Dehmlow et al., Israel J. Chem., 26, 219-221 (1985)) or lead oxide. In one example, the thioureas can be prepared from the corresponding isothiocyanate and substituted 2-hydroxyaniline. Subsequently, the concomitant ring closure with mercuric oxide to afford the substituted 2-aminobenzoxazole occurs in a one-pot two-step procedure (Garin et al., J. Heterocyclic Chem., 28, 359-363 (1991)). Polyphosphate ester has also been used to perform a similar ring closure reaction on N-(2-hydroxyphenyl)ureas (Katsura et al., Chem. Pharm. Bull., 40 (6), 1424-1438 (1992)). Displacement of a 2-chloro (J. Med. Chem., 41 (16), 3015-3021 (1998)), 2-aryloxy (Kover et al., Synthesis, 1124-1126 (1994)) or a 2-thio (Pharmazie, 1997, 52(8), 585-589) substituent on the benzoxazole or benzimidazole with nitrogen nucleophiles, to afford the 2-amino variant, has also been reported.

The main disadvantages of these methods are as follows:

a). Often the procedures require a dedicated multi-step synthesis of an intermediate thiourea or a 2-substituted benzoxazole or benzimidazole. In the majority of the procedures, these intermediates often require an elaborate work-up and/or purification prior to the final step in the synthesis of the desired product.

b). Many of the reactions require high temperatures (>100° C.) and the presence of high boiling solvents in order to proceed to completion. This can be problematic when the cyclodesulfurization step takes place in the presence of thermodynamically labile functional groups.

c). A number of the reagents present toxicological and physical hazards. For example, mercuric oxide is highly toxic (oral LD₅₀=18 mg/kg in rats) and potassium superoxide presents an explosive risk in the presence of minor organic contaminants (Bretherick, Chem. Br., 14(9), 426 (1978)).

d). In some cases, the removal of the reagent byproducts, e.g. in reactions using N,N′-dicyclohexylcarbodiimide or polyphosphate ester, can be problematic, labor intensive and unsuitable for scale-up.

### SUMMARY OF THE INVENTION

In one embodiment, the present invention is directed to a method of making an optionally substituted 2-amino-benzoxazole or 2-amino-benzimidazole which comprises reacting a corresponding optionally substituted N-(2-hydroxyphenyl)thiourea or N-(2-aminophenyl)thiourea, respectively, with a transition metal in its I or II oxidation state, in the presence or absence of a base to obtain the optionally substituted 2-amino-benzoxazole or 2-aminobenzimidazole.

In another embodiment, the present invention is directed to a process for the synthesis of a compound of formula (II),

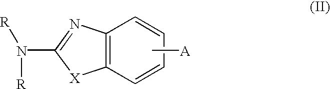

comprising the step of reacting a compound of formula (I),

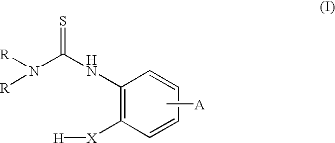

with a transition metal in its I or II oxidation state and optionally a base until the reaction is substantially complete to obtain the compound of formula (II); wherein:

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In another embodiment, the present invention is directed to a process for the synthesis of a compound of formula (II),

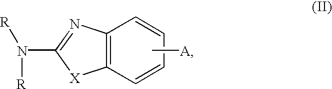

comprising the step of reacting an isothiocyanate, an optionally substituted 2-(X)-aniline, a transition metal in its I or II oxidation state and optionally a base, until the reaction is substantially complete to obtain a compound of formula (II) wherein

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and

X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In preferred embodiment, the present invention is directed to a process wherein the isothiocyanate is of the formula R—NCS and the optionally substituted aniline is of the formula

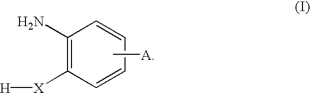

In another embodiment, the present invention is directed to a process for the synthesis of a compound of formula (II),

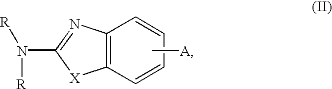

comprising the steps:

forming an isothiocyanate in situ by reacting an amine or an aniline with a reagent having a thiocarbonyl moiety and which is capable of a double nucleophilic attack at

the carbon of the thiocarbonyl moiety to yield the isothiocyanate; reacting the isothiocyanate with an optionally substituted 2-(X)-aniline, a transition metal in its I or II oxidation state and optionally a base, until the reaction is substantially complete to obtain a compound of formula (II), wherein

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and

X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In a preferred embodiment of any of the foregoing methods, the base is one or more organic bases wherein the organic base is triethylamine or ammonia, or a combination thereof.

In a preferred embodiment of any of the foregoing methods, the base is one or more inorganic bases wherein the organic base is sodium hydroxide, sodium hydrogen carbonate or cesium carbonate, or a combination thereof.

In a preferred embodiment of any of the foregoing methods, the transition metal is Cr, Mn, Fe, Co, Cu or Zn, or a combination thereof.

In a preferred embodiment of any of the foregoing methods, the transition metal is a corresponding salt or a combination of salts.

In an even more preferred embodiment of any of the foregoing methods, the transition metal salt is one or more copper salts.

In an even more preferred embodiment of any of the foregoing methods, the copper salt is copper (II) sulfate, anhydrous copper (II) sulfate or copper (I) chloride, or a combination thereof.

In an even more preferred embodiment of any of the foregoing methods, the transition metal is copper (II) sulfate, anhydrous copper (II) sulfate or copper (I) chloride, or a combination thereof.

A preferred embodiment of any of the foregoing methods, wherein the transition metal is Cr, Mn, Fe, Co, Cu or Zn, or a combination thereof and wherein the base is selected from the group consisting of triethylamine, ammonia, sodium hydroxide, sodium hydrogen carbonate and cesium carbonate.

A more preferred embodiment of any of the foregoing inventions wherein the transition metal is a corresponding salt or a combination of salts.

A more preferred embodiment of any of the foregoing inventions wherein the transition metal is selected from the group consisting of copper (II) sulfate, anhydrous copper (II) sulfate or copper (I) chloride.

In another embodiment, the present invention is directed to a method of making an optionally substituted 2-amino-benzoxazole or 2-amino-benzimidazole which comprises reacting a corresponding optionally substituted N-(2-hydroxyphenyl)thiourea or N-(2-aminophenyl)thiourea, respectively, with a transition metal in its III oxidation state, in the presence or absence of a base to obtain the optionally substituted 2-amino-benzoxazole or 2-aminobenzimidazole.

In another embodiment, the present invention is directed to a process for the synthesis of a compound of formula (II),

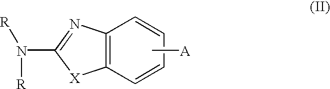

comprising the step of reacting a compound of formula (I),

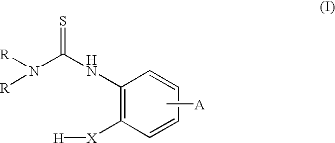

with a transition metal in its III oxidation state and optionally a base until the reaction is substantially complete to obtain the compound of formula (II); wherein:

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In another embodiment, the present invention is directed to a process for the synthesis of a compound of formula (II),

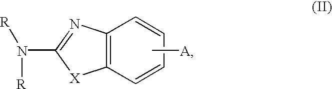

comprising the step of reacting an isothiocyanate, an optionally substituted 2-(X)-aniline, a transition metal in its III oxidation state and optionally a base, until the reaction is substantially complete to obtain a compound of formula (II) wherein

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and

X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In a preferred embodiment of any of the foregoing methods pertaining to using a transition metal in its III oxidation state, wherein the isothiocyanate is of the formula R—NCS and the optionally substituted aniline is of the formula

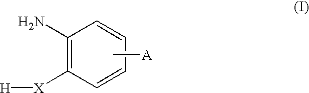

In another embodiment of any of the foregoing methods pertaining to using a transition metal in its III oxidation state, the present invention is directed to the synthesis of a compound of formula (II),

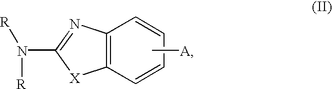

comprising the steps:

forming an isothiocyanate in situ by reacting an amine or an aniline with a reagent having a thiocarbonyl moiety and which is capable of a double nucleophilic attack at the carbon of the thiocarbonyl moiety to yield the isothiocyanate;

reacting the isothiocyanate with an optionally substituted 2-(X)-aniline, a transition metal in its III oxidation state and optionally a base, until the reaction is substantially complete to obtain a compound of formula (II), wherein

A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl;

Z² is an optionally substituted alkyl, aryl, heterocyclyl, arylalkyl, or heterocyclylalkyl;

R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and

X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

In a preferred embodiment of any of the foregoing methods, pertaining to using a transition metal in its III oxidation state, the base is one or more organic bases wherein the organic base is triethylamine or ammonia, or a combination thereof.

In a preferred embodiment of any of the foregoing methods pertaining to using a transition metal in its m oxidation state, the base is one or more inorganic bases wherein the inorganic base is sodium hydroxide, sodium hydrogen carbonate or cesium carbonate, or a combination thereof.

In a preferred embodiment of any of the foregoing methods pertaining to using a transition metal in its III oxidation state, wherein the transition metal is Cr, Mn, Fe, or Co or a combination thereof.

In an even more preferred embodiment of any of the foregoing methods pertaining to using a transition metal in its III oxidation state, wherein the transition metal is a corresponding salt or a combination of salts.

In an even more preferred embodiment of any of the foregoing methods, pertaining to using a transition metal in its III oxidation state wherein the transition metal salt is one or more iron salts.

In an even more preferred embodiment of any of the foregoing methods, pertaining to using a transition metal in its III oxidation state wherein the iron salt is iron (III) chloride, or anhydrous iron (III) chloride, or a combination thereof.

In an even more preferred embodiment of any of the foregoing methods, pertaining to using a transition metal in its III oxidation state wherein the transition metal salt is manganese (III) acetate dihydrate or anhydrous manganese (III) diacetate, iron (III) chloride, anhydrous iron (III) chloride or a combination thereof.

In an even more preferred embodiment of any of the foregoing methods, pertaining to using a transition metal in its III oxidation state wherein the transition metal is manganese (III) acetate dihydrate or anhydrous manganese (III) diacetate, or a combination thereof.

A preferred embodiment of any of the foregoing methods pertaining to using a transition metal in its III oxidation state wherein the transition metal is Cr, Mn, Fe, or Co or a combination thereof and wherein the base is selected from the group consisting of triethylamine, ammonia, sodium hydroxide, sodium hydrogen carbonate and cesium carbonate.

A more preferred embodiment of any of the foregoing methods pertaining to using a transition metal in its III oxidation state wherein the transition metal is a corresponding salt.

An even more preferred embodiment of any of the foregoing methods wherein the transition metal salt is selected from the group consisting of iron (III) chloride, anhydrous iron (III) chloride, manganese (III) acetate dihydrate and anhydrous manganese (III) diacetate.

A preferred embodiment of any of the foregoing methods wherein the base is absent.

### DETAILED DESCRIPTION OF THE INVENTION

This invention relates to a novel transition metal mediated process for the preparation of optionally substituted 2-amino-benzoxazoles and 2-amino-benzimidazoles. In one aspect, the process is useful for preparing optionally substituted 2-amino-benzoxazoles or 2-amino-benzimidazoles which are useful as drugs such as kinase inhibitors or as intermediates for making other compounds that are useful as drugs.

The invention particularly relates to the use of a transition metal, preferably as a salt, for example iron, manganese or copper salts, particularly iron (III) chloride, anhydrous copper (II) sulfate, or manganese (II) acetate dihydrate, optionally in the presence of a base, e.g. triethylamine, and preferably in the presence of the base, as highly active reagents for the desulfurization and concomitant ring closure of optionally substituted N-(2-hydroxyphenyl)thioureas or N-(2-aminophenyl)thioureas to afford the corresponding optionally substituted 2-amino-benzoxazole or 2-amino-benzimidazole, respectively.

The process offers the advantages that it can be performed under mild temperatures, for example about to 20° C. to about 60° C., preferably 20° C., however higher and lower temperatures can be used, and in a range of organic solvents, for example tetrahydrofuran, acetonitrile and dichloromethane. The substituted N-(2-hydroxyphenyl)thioureas or N-(2-aminophenyl)thioureas can be prepared from the corresponding isothiocyanate and either the substituted or unsubstituted 2-amino phenol or the substituted or unsubstituted phenylenediamine, respectively, in a single-pot reaction.

In a variation of the above procedure, the isothiocyanate can be formed in situ in the reaction vessel from either an amine or an aniline using reagents known in the art for making isothiocyanates, for example, and not exclusively including, 1,1′-thiocarbonyldi-2-(1H)pyridone, 1,1′-thiocarbonyldiimidazole or thiophosgene. Once this reaction is complete, the remaining reagents may be added to the same reaction vessel according to the general procedure described herein to afford the optionally substituted 2-amino-benzoxazole or 2-amino-benzimidazole in a single pot procedure.

Additionally, the iron, manganese or copper salts and a base can be added, simultaneously, with the isothiocyanate and the substituted aniline to afford the optionally substituted 2-amino-benzoxazole and 2-amino-benzimidazole in a one-pot, one-step procedure.

Copper salts offer the advantages of low cost and low toxicity, for example, copper (II) sulfate has an oral LD₅₀ in rats of 300 mg/kg, and iron (III) chloride has an oral LD₅₀ in rats of 450 mg/kg.

The following terms have the noted meanings as used herein

“Alkyl” refers to a saturated aliphatic hydrocarbon, or an aliphatic group having one or more unsaturated groups, including straight-chain and branched-chain groups. Preferred straight chain and branched alkyl groups include C₁-C₈ alkyl groups.

“Alkenyl” refers to an aliphatic hydrocarbon having at least one double bond, including straight-chain and branched-chain groups. Preferred straight chain and branched alkenyl groups include C₁-C₈ alkyl groups.

“Alkynyl” refers to an aliphatic hydrocarbon having at least one triple bond, including straight-chain and branched-chain groups. Preferred straight chain and branched alkynyl groups include C₁-C₈ alkyl groups.

“Alkoxy” refers to an “O-alkyl” group, where “alkyl” is defined as described above.

“Cycloalkyl” refers to mono-, bi- and tri-carbocyclic groups having 3 to 12 carbon atoms, preferred cycloalkyl groups have 3 to 6 ring carbon atoms.

“Heterocyclyl” means an optionally substituted mono- or bi-cyclic aromatic or non-aromatic heterocycle in which the heterocycle contains 1, 2, 3 or 4 hetero atoms selected from nitrogen, sulphur or oxygen. The heterocyclyl group may be attached through a carbon atom or a hetero atom. Suitable heterocyclyl groups include but are not restricted to 1,3-dioxolanyl, 1,4-dioxolanyl, morpholinyl, piperidinyl, piperazinyl, thiomorpholinyl, 3H-indolyl, 4H-quinolizinyl, 2-imidazolinyl, imidazolidinyl, quinuclidinyl, 2-pyrazolinyl, pyrazolidinyl, 2H-pyranyl, 4H-pyranyl, 1,4-dithianyl, 1,3,5-trithianyl, tetrahydrofuranyl, pyrrolidinyl, pyrrolyl, imidazolyl, isothiazolyl, pyrazolyl, thiazolyl, oxazolyl, oxadiazolyl, thiadiazolyl, tetrazolyl, pyridyl, pyridazinyl, pyrimidinyl, pyrazinyl, benzimidazolyl, quinolinyl, isoquinolinyl, indazolyl, furanyl, 2,3,4,5-tetrahydrofuranyl, thienyl, benzofuranyl, indolizinyl, imidazopyridinyl, isoxazolyl, benzoxazolyl, indolyl, isoindolyl, indolinyl, benzothiazolyl, benzothienyl, purinyl, 1,2,3-triazolyl, 1,2,4-trizolyl, 1,3,5-triazinyl, cinnolinyl, phthalazinyl, quinazolinyl, quinoxalinyl, 1,8-naphthypyridinyl, pteridinyl, carbazolyl, acridinyl, phenazinyl, phenothiazinyl and phenoxazinyl.

“Aryl” means a mono-, bi- or tri-cyclic aromatic group. Suitable aryl groups include phenyl, indenyl, naphthyl, azulenyl, fluorenyl and anthracenyl.

The term “optionally substituted” means that the moiety that it modifies can be substituted with any one or more substituents known to one skilled in the art that results in a chemically stable molecule. Since the methods of the present invention are not limited by the substituents attached to the starting material isothiocyanate and 2-(X)-aniline, all such compounds are within the scope of the present invention. Preferred substituents within the “optionally substituted” non-exclusively includes: halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;

where R¹ for each occurrence is independently H, or optionally substituted alkyl, heterocyclyl, aryl, aralkyl or heterocyclylalkyl;

p is 1 or 2;

R for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—-O-alkyl, —W—(CH₂)ₜ—S-alkyl, or —W—(CH₂)ₜ—OH;

R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;

W is a covalent bond, O, S, S(O), S(O)₂ or NR^{f}, where R^{f} is H or alkyl;

t for each occurrence is independently an integer from 2 to 6;

Z¹ is a covalent bond or alkyl; and

Z² is an optionally substituted alkyl, aryl, heterocyclyl or arylalkyl, or heterocyclylalkyl.

Unless otherwise specified, all starting materials and solvents were obtained from commercially available sources and were used without further purification. Further starting materials can be synthesized according to known literature methods or according to the skills of one of ordinary skill in the art.

All articles and patents cited in the present application are incorporated herein by reference in their entirety.

One embodiment of a process of the present invention is described in Scheme 1.

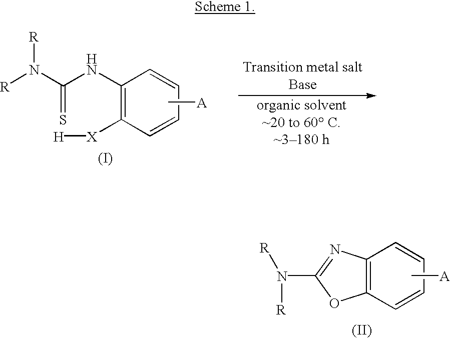

wherein the variables are as defined hereinabove.

Transition metal=Cr, Mn, Fe, Co, Cu or Zn, salts thereof or a combination of the aforementioned metals, wherein the metal is in its I, II or III oxidation state. Preferred are the salts of the foregoing metals or combination thereof.

Base=an organic base, for example, triethylamine or ammonia, or an inorganic base, for example, sodium hydroxide or sodium hydrogen carbonate.

The starting thiourea (I) is subjected to cyclodesulfurization using a transition metal, as noted above, preferably in the form of a salt, for example anhydrous iron (III) chloride, copper (II) sulfate, manganese (II) acetate dihydrate or copper (I) chloride, and an organic or inorganic base, preferably an organic base, for example triethylamine, to afford the corresponding optionally substituted 2-aminobenzoxazoles or 2-aminobenzimidazoles (II). The ring closure reaction can take place in a range of organic solvents, preferably in one or a mixture of non-protic solvents, in particular tetrahydrofuran, acetonitrile, and dichloromethane, and at mild temperatures, typically about 20° C. to about 60° C. The reaction provides good to excellent yields of the desired product within this temperature range. However, temperatures outside of the range may be utilized to obtain the desired product. In general, the reaction proceeds faster at higher temperatures within the range of 20° C. to 60° C.

For transition metals in the I or II oxidation state, the reaction performs efficiently in the presence or absence of silica. Further, reducing the stoichiometry of the transition metal, for example 1.1 equivalents, has no affect on the reaction yield yet significantly helps facilitate the reaction work-up and purification procedures.

In addition to salt forms of a transition metal used in a process of the present invention, other forms of transition metals that can be used in a process of the present invention include complexes of a transition metal and a resin or support bound transition metal. An example of a transition metal complex is [Cu(OH)(N,N,N′N′-tetramethylethylenediamine)]₂Cl₂ (Collman et al; Org. Lett, 9(2), 1233-1236, (2000) and J. Org. Chem., 66, 1528, (2001)) which can be used as a catalyst for the cyclodesulfurization reaction. For an example of a transition metal such as a copper reagent bound to a solid support or polymer, see: Amaratunga et al., Polym. Prepr., 22(1), 151-2, (1981), Kalalova et al., Collect. Czech. Chem. Commun., 48(7), 2021-7, (1983), and Koning et al., React. Polym., Ion Exch., Sorbents 4(4), 293-309, (1986).

The starting thiourea (I) is subjected to cyclodesulfurization using a transition metal, as noted above, preferably in the form of a salt, for example anhydrous iron (III) chloride or manganese (II) acetate dihydrate, and an organic or inorganic base, preferably an organic base, for example triethylamine, to afford the corresponding optionally substituted 2-aminobenzoxazoles or 2-aminobenzimidazoles (II). The ring closure reaction can take place in a range of organic solvents, preferably in one or a mixture of non-protic solvents, in particular tetrahydrofuran, acetonitrile, and dichloromethane, and at mild temperatures, typically about 20° C. to about 60° C. The reaction provides good to excellent yields of the desired product within this temperature range. However, temperatures outside of the range may be utilized to obtain the desired product.

In addition to salt forms of a transition metal used in a process of the present invention, other forms of transition metals that can be used in a process of the present invention include complexes of a transition metal and a resin or support bound transition metal.

The following Scheme 2 illustrates a method for obtaining an intermediate thiourea of formula (I), where the variables are as defined hereinabove and one R is hydrogen:

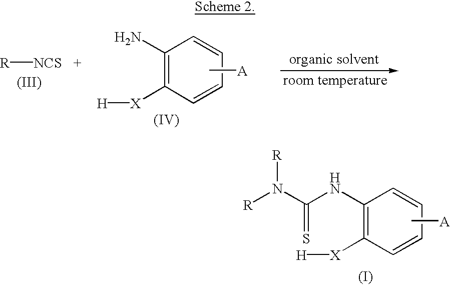

The thioureas (I) can be prepared from the corresponding isothiocyanate (III) and the 2-substituted aniline (IV). Once the thiourea (I) formation is complete, the reaction illustrated in Scheme 1 can be carried out in the same reaction vessel without having to isolate and purify the thiourea (I). For example by adding a transition metal as described hereinabove, such as iron (III) chloride, manganese (II) acetate dihydrate, anhydrous copper (II) sulfate, or copper (I) chloride and a base, such as triethylamine, to the crude reaction mixture to afford the corresponding optionally substituted 2-amino-benzoxazole or 2-amino-benzimidazole product of formula (II) in a one-pot, two-step procedure. Thus, the intermediate thiourea (I) does not require isolation or purification during this process.

In another embodiment, a transition metal, preferably a salt thereof, for example iron (III) chloride, manganese (II) acetate dihydrate, copper (II) sulfate, or copper (I) chloride, and a base, e.g. triethylamine, can be added simultaneously with the isothiocyanate (III) to an optionally substituted 2-X aniline (IV) to afford the corresponding optionally substituted 2-amino-benzoxazole or 2-amino-benzimidazole of formula (II), in a one-pot, one-step procedure.

Furthermore, a starting material isothiocyanate can be formed in situ in the reaction vessel from either an amine or an aniline using reagents known in the art, for example, and not exclusively including, 1,1′-thiocarbonyldi-2-(1H)pyridone, 1,1′-thiocarbonyldiimidazole or thiophosgene. Once this reaction is complete, the remaining reagents may be added to the same reaction vessel according to the general procedure described herein to afford the corresponding optionally substituted 2-amino-benzoxazole or 2-amino-benzimidazole in a single pot procedure.

An optionally substituted 2-amino-benzoxazole or 2-amino-benzimidazole of formula (II) can be isolated according to standard methods known in the art. For example, by removing the reaction solvent in vacuo, dissolving the residue in an organic solvent, for example, ethyl acetate, diethyl ether or dichloromethane, and washing with aqueous solutions, known to those skilled in the art, which can sequester the transition metal, such as a copper salt, for example, these include: aqueous solutions of ammonia, picolinic acid, oxalic acid, pyridine, and ethylenediaminetetraacetic acid (EDTA) or corresponding salts (Parker et al., EP 0740015). The product can then be subjected to additional purification, using methods such as filtration, recrystallization or chromatography, as desired. In those embodiments of the present invention wherein a complex of a transition metal or a transition metal bound to a solid support is used in the reaction, various isolation and purification methods for obtaining the desired optionally substituted 2-amino-benzoxazole or 2-amino-benzimidazole, for example, filtration, are known to those skilled in the art.

The following examples serve to illustrate the present invention and are not to be construed as limiting the scope of the present invention to the embodiments so exemplified. Nuclear magnetic resonance (NMR) were measured on a 400 MHz Bruker instrument and peak positions are expressed in parts per million (ppm). The peak shapes are denoted as follows: s, singlet; d, doublet; dd, double doublet; t, triplet; hept, heptet; m, multiplet. “J” denotes the splitting constant which is expressed in Hertz (Hz).

### EXAMPLE 1

### N2-(4-Bromophenyl)-5-trifluoromethyl-1,3-benzoxazol-2-amine

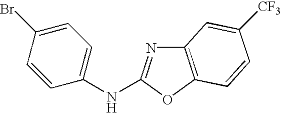

4-Bromophenyl isothiocyanate (1.667 g, 7.785 mmol) was added to a solution of 2-amino-4-trifluoromethylphenol (1.379 g, 7.785 mmol) in tetrahydrofuran (THF) (100 mL) and the reaction was stirred at room temperature for about 16 hours then at about 50° C. for about another 5 hours. Copper (I) chloride (0.771 g, 7.785 mmol) and triethylamine (1.08 mL, 7.785 mmol) were added, and the mixture was stirred at room temperature for about 72 hours and then at about 50° C. for about another 18 hours. Additional copper (I) chloride (0.385 g) was added and the reaction was stirred at about 60° C. for about another 2 hours. The reaction was concentrated under reduced pressure, dissolved in methanol (200 mL), filtered through a pad of diatomaceous earth and the solvent removed in vacuo to afford N2-(4-bromophenyl)-5-trifluoromethyl-1,3-benzoxazol-2-amine as a brown solid (3.90 g, 140% of theory); RP-HPLC Rt 17.627 min, 77% purity (5% to 85% acetonitrile/0.1M aqueous ammonium acetate, buffered to pH 4.5, over 20 min at 1 mL/min; λ=254 nm; Waters Deltapak® C18, 300 Å, 5 μm, 150×3.9 mm column); and m/z 354.9 and 356.9 (M-H)⁻.

### EXAMPLE 2

### N2-(4-Bromophenyl)-5-methyl-1,3-benzoxazol-2-amine

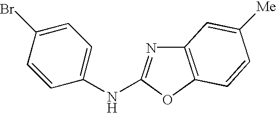

4-Bromophenyl isothiocyanate (2.0 g, 9.34 mmol) was added to a solution of 2-amino-4-methylphenol (1.15 g, 9.34 mmol) in acetonitrile (100 mL) and the reaction was stirred at room temperature for about 16 hours. The formation of the intermediate N-(4-bromophenyl)-N′-(2-hydroxy-5-methylphenyl)thiourea was complete, as analyzed by RP-HPLC Rt 13.010 min, 98% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column). Copper (I) chloride (0.925 g, 9.34 mmol) and triethylamine (1.29 mL, 9.34 mmol) were added, and the mixture was stirred at room temperature for about 6 days. The reaction was concentrated under reduced pressure, dissolved in methanol (200 mL), filtered through a pad of diatomaceous earth and the solvent removed in vacuo to afford a brown solid. The solid was dissolved in dichloromethane (200 mL), washed with water (2×200 mL), dried over anhydrous sodium sulfate and absorbed onto silica (10 mL). The product was purified by chromatography through a silica pad using 10% ethyl acetate in n-heptane as the eluent to afford N2-(4-bromophenyl)-5-methyl-1,3-benzoxazol-2-amine as a yellow solid (0.30 g, 11%); RP-HPLC Rt 16.451 min, 95% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); and m/z 302.9 and 304.9 (MH⁺).

### EXAMPLE 3

### N2-(4-Bromophenyl)-5-methyl-1,3-benzoxazol-2-amine

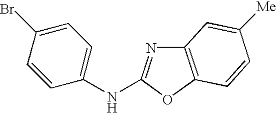

4-Bromophenyl isothiocyanate (2.0 g, 9.34 mmol) was added to a solution of 2-amino-4-methylphenol (1.15 g, 9.34 mmol) in tetrahydrofuran (100 mL) and the reaction was stirred at room temperature for about 16 hours. The formation of the intermediate N-(4-bromophenyl)-N′-(2-hydroxy-5-methylphenyl)thiourea was complete, as analyzed by RP-HPLC Rt 12.973 min, 88% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column). Anhydrous copper (II) sulfate (14.06 g, 88.10 mmol), silica gel (14.06 g), and triethylamine (1.3 mL, 9.34 mmol) were added, and the mixture was stirred at room temperature for about another 72 hours. The reaction was filtered through a pad of diatomaceous earth washed with diethyl ether (3×100 mL) and the combined filtrate was concentrated under reduced pressure to afford N2-(4-bromophenyl)-5-methyl-1,3-benzoxazol-2-amine as a brown solid (2.70 g, 95%); RP-HPLC Rt 16.433 min, 99% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); and ¹H NMR (400 MHz, d₆-DMSO) 2.37 (3H, s), 6.94 (1H, d, J 8.1 Hz), 7.27 (1H, s), 7.36 (1H, d, J 8.1 Hz), 7.54 (2H, d, J 8.4 Hz), 7.72 (2H, d, J 8.4 Hz), and 10.72 (1H, s).

### EXAMPLE 4

### N2-(4-Bromophenyl)-7-isopropyl-1,3-benzoxazol-2-amine

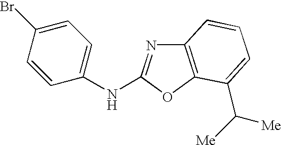

4-Bromophenyl isothiocyanate (0.50 g, 2.34 mmol) was added to a solution of 2-amino-6-isopropylphenol (0.354 g, 2.34 mmol) in tetrahydrofuran (35 mL) and the reaction was stirred at room temperature for about 3 hours. Anhydrous copper (II) sulfate (3.361 g, 21.06 mmol), silica gel (3.361 g), and triethylamine (0.33 mL, 2.34 mmol) were added, and the mixture was stirred at room temperature for about 18 hours. The reaction was filtered through a pad of diatomaceous earth, the diatomaceous earth was washed with diethyl ether (3×50 mL), and the combined filtrate was concentrated under reduced pressure and the resulting brown solid was purified by column chromatography through a silica pad using neat ethyl acetate as the eluent to afford N2-(4-bromophenyl)-7-isopropyl-1,3-benzoxazol-2-amine as a light brown solid (0.70 g, 91%); RP-HPLC Rt 18.066 min, 86% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); ¹H NMR (400 MHz, d₆-DMSO) 1.34 (6H, d, J 6.9 Hz), 3.25 (1 H, hept, J 6.9 Hz), 7.02 (1H, d, J 7.3Hz), 7.16 (1H, t, J 7.7 Hz), 7.29 (1H, dd, J 7.7 and 1.1 Hz), 7.55 (2H, dd, J 6.9 and 2.1 Hz), 7.74 (2H, dd, J 6.9 and 2.1 Hz) and 10.807 (1H, s).

### EXAMPLE 5

### N2-(4-Bromophenyl)-5-cyano-1,3-benzoxazol-2-amine

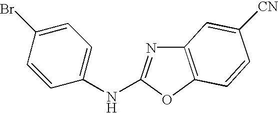

4-Bromophenyl isothiocyanate (2.93 g, 0.0137 mol) was added to a solution of 3-amino-4-hydroxybenzonitrile (1.84 g, 0.0137 mol) in acetonitrile (140 mL) at room temperature. The mixture was stirred for about 16 hours prior to the addition of copper (I) chloride (1.36 g, 0.0137 mol) and triethylamine (1.9 mL, 0.0137 mol). The mixture was stirred for about another 16 hours and the solvent was removed under reduced pressure. The solid was dissolved in methanol (100 mL), filtered through a pad of diatomaceous earth, and washed with additional methanol (2×50 mL). The brownish filtrate was left to stand at about 4° C. for about 3 days and the resulting precipitate was collected by filtration to afford N2-(4-bromophenyl)-5-cyano-1,3-benzoxazol-2-amine (2.4 g, 0.0076 mol, 55%); RP-HPLC Rt 11.1 min, 92% purity (Delta Pak C18, 5 μm, 300 Å, 15 cm; 5%-95% acetonitrile-0.1M ammonium acetate over 10 min, 1 mL/min); and ¹H NMR (400 MHz, d₆-DMSO) 7.59 (3H, m), 7.72 (3H, m), 7.97 (1H, s), and 11.12 (1H, s).

### EXAMPLE 6

### N2-(4-Bromophenyl)-5-(trifluoromethoxy)-1,3-benzoxazol-2-amine

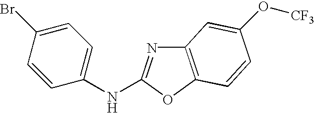

4-Bromophenyl isothiocyanate (1.00 g, 0.0047 mol) was added to a solution of 2-amino-4-(trifluoromethoxy)phenol (0.90 g, 0.0047 mol) in tetrahydrofuran (60 mL) at room temperature. The mixture was stirred for about 16 hours prior to the addition of anhydrous copper (II) sulfate (7.10 g, 0.0443 mol), triethylamine (0.67 mL, 0.0047 mol) and silica gel (8.50 g). The mixture was stirred for about another 4 hours and the solvent was then removed under reduced pressure. The residue was purified by column chromatography through a silica pad using 25% ethyl acetate in n-heptane as the eluent. The resulting orange solid was further purified by chromatography over silica gel; using a 0% to 25% ethyl acetate in n-heptane gradient as the eluent. The solid was triturated with n-heptane to give N2-(4-bromophenyl)-5-(trifluoromethoxy)-1,3-benzoxazol-2-amine (0.90 g, 0.0024 mol, 51%); RP-HPLC Rt 12.2 min, 99% purity (DeltaPak® C18, 5 μm, 300 Å, 15 cm; 5%-95% acetonitrile-0.1M ammonium acetate over 10 min, 1 mL/min); and m/z 373 and 375 (MH⁺).

### EXAMPLE 7

### N2-(4-Bromophenyl)-5-ethyl-1,3-benzoxazol-2-amine

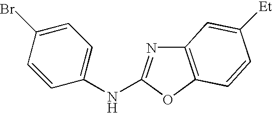

4-Bromophenyl isothiocyanate (1.40 g, 0.0065 mol) was added to a solution of 2-amino-4-ethylphenol (0.89 g, 0.0065 mol) in tetrahydrofuran (80 mL) at room temperature. The mixture was stirred for about 2 hours prior to the addition of anhydrous copper (II) sulfate (6.2 g, 0.039 mol), triethylamine (0.9 mL, 0.0065 mol) and silica gel (11.7 g). The mixture was stirred for an another 4 hours and the solvent was then removed under reduced pressure. The residues were purified by column chromatography through a silica pad using 25% ethyl acetate in n-heptane as the eluent. The resulting brown solid was further purified by chromatography over silica gel; using a 0% to 25% ethyl acetate in n-heptane gradient as the eluent. The solid was triturated with n-heptane to give N2-(4-bromophenyl)-5-ethyl-1,3-benzoxazol-2-amine (0.96g, 0.003 mol, 46%); RP-HPLC Rt 12.1 min, 99% purity (DeltaPak® C18, 5 μm, 300 Å, 15 cm; 5%-95% acetonitrile/0.1M ammonium acetate over 10 min, 1 mL/min); and m/z 317 and 319 (MH⁺).

### EXAMPLE 8

### N2-(4-Bromophenyl)-5,7-dimethyl-1,3-benzoxazol-2-amine

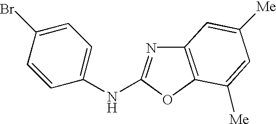

2-Amino-4,6-dimethylphenol (0.214 g, 1.00 mmol) was added to a solution of 4-bromophenyl isothiocyanate (0.137 g, 1.00 mmol) in tetrahydrofuran (15 mL) and the reaction was stirred at room temperature for about 12 hours. Anhydrous copper (II) sulfate (1.50 g, 9.43 mmol), silica gel (1.50 g), and triethylamine (0.14 mL, 1.00 mmol) were added, and the mixture was stirred at room temperature for about another 16 hours. The reaction was filtered through a pad of diatomaceous earth, washed with additional tetrahydrofuran (2×20 mL), and the combined filtrate was concentrated under reduced pressure to afford N2-(4-bromophenyl)-5,7-dimethyl-1,3-benzoxazol-2-amine as a brown pink solid (0.30 g, 90%); RP-HPLC Rt 17.395 min, 95% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); ¹H NMR (400 MHz, d₆-DMSO) 2.33 (3H, s), 2.38 (3H, s), 6.79 (1H, s), 7.09 (1H, s), 7.54 (2H, dd, J 11.7 and 2.9 Hz), 7.72 (2H, dd, J 11.7 and 2.9 Hz) and 10.77 (1H, s).

Examples Detailing the Range of Reaction Conditions for the Synthesis of N2-(4-Bromophenyl)-5-methyl-1,3-benzoxazol-2-amine

The following reaction conditions serve to illustrate the range of viable conditions and are not to be construed as limiting the scope of the present invention to the protocols exemplified.

i). Optimization of Ratios of Reagents and Temperature

### EXAMPLES 8.1 TO 8.8

2-Amino-4,6-dimethylphenol (1-10 mmol) was added to a solution of a substituted 4-bromophenyl isothiocyanate (1 equivalent) in tetrahydrofuran (20 100 mL) and the reaction was stirred at room temperature for about 2-24 hours. Once the formation of the intermediate, N-(4-bromophenyl)-N′-(2-hydroxy-3,5-dimethylphenyl)thiourea, was complete, anhydrous copper (II) sulfate (0-10 equivalents), silica gel (0-30 equivalents) and triethylamine (1 equivalent) were added and the reaction mixture was stirred at a temperature between about 20 and about 60° C. for about 3-93 hours. The reaction was worked-up using one of the following procedures:

A. The reaction mixture was filtered through a pad of diatomaceous earth, washed with additional tetrahydrofuran (2×20 mL), and the combined filtrate was concentrated under reduced pressure. The residue was dissolved in ethyl acetate (400 mL) and washed with one of the following:

A.1. 10% w/v aqueous EDTA (3×100 mL)

A.2. 10% v/v aqueous pyridine (3×100 mL)

A.3. 10% v/v aqueous ammonium hydroxide (28 to 30% ammonia content) (3×100 mL).The organic layer was then dried over anhydrous magnesium sulfate and concentrated under reduced pressure to afford N2-(4-bromophenyl)-5-methyl-1,3-benzoxazol-2-amine as a brown-pink solid

B. The reaction mixture was concentrated under reduced pressure then added to a silica pad. Purification by chromatography using 17% ethyl acetate in n-heptane (2 L), followed by diethyl ether as the eluent gave N2-(4-bromophenyl)-5-methyl-1,3-benzoxazol-2-amine.

C. The reaction mixture was filtered through a pad of diatomaceous earth, washed with additional tetrahydrofuran (2×20 mL), and the combined filtrate was concentrated under reduced pressure to give N2-(4-bromophenyl)-5-methyl-1,3-benzoxazol-2-amine.

A summary of the results are detailed in Table 1.

TABLE 1
Examples of different reaction conditions and work-up protocols employed

| | | | | | | | | | Isolated Yield (%) |
|---|---|---|---|---|---|---|---|---|---|
| | | Equiv. of | | | | | | | (HPLC % purity) |
| | Scale | copper (II) | Equiv. | Equiv. of | | Temper- | Reaction | Work-up | ((Cu content |
| Entry | (mmol) | sulfate | of silica | triethylamine | Solvent | ature (° C.) | time (h) | procedure | (ppm))) |
| 8.1 | 2,4-Dimethyl- | 9.43 | 30 | 1.0 | THF | RT | 24 | A.1 | 72% |
| | 6-aminophenol | | | | | | | | (94% purity) |
| | (3.11) | | | | | | | | |
| 8.2 | 2,4-Dimethyl- | 9.43 | 30 | 1.0 | THF | RT | 24 | A.2 | 77% |
| | 6-aminophenol | | | | | | | | (94% purity) |
| | (3.11) | | | | | | | | ((875 ppm)) |
| 8.3 | 2,4-Dimethyl- | 9.43 | 30 | 1.0 | THF | RT | 24 | A.3 | 74% |
| | 6-aminophenol | | | | | | | | (94% purity) |
| | (3.11) | | | | | | | | ((708 ppm)) |
| 8.4 | 2,4-Dimethyl- | 1.0 | 30 | 1.0 | THF | RT | 168 | A.3 | 76% |
| | 6-aminophenol | | | | | | | | (93% purity) |
| | (2.13) | | | | | | | | |
| 8.5 | 2,4-Dimethyl- | 1.1 | 0 | 1.0 | CH₃CN | RT | 27 | A.3 | 70% |
| | 6-aminophenol | | | | | | | | (>99.5% purity) |
| | (2.36) | | | | | | | | |
| 8.6 | 2,4-Dimethyl- | 1.1 | 0 | 1.0 | CH₂Cl₂ | RT | 49 | A.3 | 74% |
| | 6-aminophenol | | | | | | | | (95% purity) |
| | (2.36) | | | | | | | | |
| 8.7 | 2,4-Dimethyl- | 1.1 | 0 | 1.0 | THF | RT | 49 | A.3 | 70% |
| | 6-aminophenol | | | | | | | | (87% purity) |
| | (1.17) | | | | | | | | |
| 8.8 | 2,4-Dimethyl- | 1.1 | 0 | 1.0 | THF | 60 | 3 | A.3 | 99% |
| | 6-aminophenol | | | | | | | | (91% purity) |
| | (1.17) | | | | | | | | ((178 ppm)) |

RP-HPLC conditions used: (5% to 85% acetonitrile/0.1 M aqueous ammonium acetate, buffered to pH 4.5, over 20 min at 1 mL/min; λ = 254 nm; Deltapak ® C18, 300 Å, 5 μm, 150 × 3.9 mm column).

### EXAMPLE 8.9.

2-Amino-4,6-dimethylphenol (0.160 g, 1.17 mmol) anhydrous copper (II) sulfate (0.21 g, 1.29 mmol) and triethylamine (0.164 mL, 1.17 mmol) were added to a solution of 4-bromophenyl isothiocyanate (0.250 g, 1.17 mmol), in tetrahydrofuran (20 mL) and the reaction was stirred at room temperature for about 24 hours. The reaction was filtered through a pad of diatomaceous earth, washed with ethyl acetate (2×20 mL), and the combined filtrate was concentrated under reduced pressure to afford N2-(4-bromophenyl)-5,7-dimethyl-1,3-benzoxazol-2-amine as a brown pink solid (0.33 g, 89%); RP-HPLC Rt 17.294 min, 93% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column).

### EXAMPLE 8.10

Using the same scale and reaction procedure as detailed in Example 8.9, except that the reaction was stirred at about 60° C. for about 22 hours, N2-(4-bromophenyl)-5,7-dimethyl-1,3-benzoxazol-2-amine was afforded as a brown-pink solid (0.34 g, 91%); RP-HPLC Rt 17.268 min, 90% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column).

### EXAMPLE 9

### N2-(3-Pyridyl)-1,3-benzoxazol-2-amine

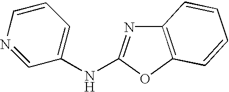

3-Pyridyl isothiocyanate (0.311 g, 2.29 mmol) was added to a solution of 2-aminophenol (0.250 g, 2.29 mmol) in tetrahydrofuran (15 mL) and the reaction was stirred at room temperature for about 3 hours. Anhydrous copper (II) sulfate (0.410 g, 2.52 mmol) and triethylamine (0.32 mL, 2.29 mmol) were added, and the mixture was stirred at about 60° C. for about 96 hours. The reaction was filtered through a pad of diatomaceous earth, the diatomaceous earth was washed with ethyl acetate (3×50 mL), and the combined filtrate was concentrated under reduced pressure. The residue was dissolved in methylene chloride (200 mL), washed with 10% v/v aqueous ammonium hydroxide (3×100 mL), dried over anhydrous magnesium sulfate and concentrated under reduced pressure to afford N2-(3-pyridyl)-1,3-benzoxazol-2-amine as a yellow solid (0.343 g, 71%); RP-HPLC Rt 9.580 min, 97% purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); ¹H NMR (400 MHz, d₆-DMSO) 7.19 (1H, m), 7.24 (1H, m), 7.42 (1H, m), 7.48 (2H, m), 8.27 (2H, m), 8.87 (1H, d, J 2.3 Hz) and 10.87 (1H, s).

### EXAMPLE 10

### N2-(4-Methoxyphenyl)-1,3-benzoxazol-2-amine

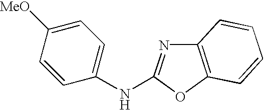

Using the protocol and scale described for the synthesis of Example 9. The cyclodesulfurization step was complete after about 72 hours at about 60° C. and purified in the same way as detailed in Example 9 to afford N2-(4-methoxyphenyl)-1,3-benzoxazol-2-amine as a brown solid (0.536 g, 97%); RP-HPLC Rt 12.529 min, 94 purity (5% to 85% acetonitrile/0.1M aqueous ammonium acetate, buffered to pH 4.5, over 20 min at 1 mL/min; λ=254 nm; Deltapak® C18, 300 Å, 5 μm, 150×3.9 mm column); ¹H NMR (400 MHz, d₆-DMSO) 3.75 (3H, s), 6.97 (2H, dd, J 6.9 and 2.2 Hz), 7.11 (1H, dt, J 6.5 and 1.1 Hz), 7.20 (1H, dt, J 6.5 and 1.1 Hz), 7.41 (1H, dd, J 7.7 and 0.6 Hz), 7.45 (1H, dd, J 7.7 and 0.6 Hz), 7.65 (2H, dd, J 6.9 and 2.2 Hz) and 10.38 (1H, s).

### EXAMPLE 11

### N2-(4-Nitrophenyl)-1,3-benzoxazol-2-amine

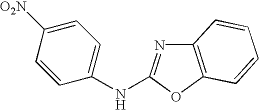

Using the protocol and scale described for the synthesis of Example 9. The cyclodesulfurization step was complete after about 22 hours at about 60° C. and purified in the same way as detailed in Example 9 to afford N2-(4-nitrophenyl)-1,3-benzoxazol-2-amine as a yellow solid (0.409 g, 70%); RP-HPLC Rt 13.876 min, >99.9% purity (5% to 85% acetonitrile/0.1M aqueous ammonium acetate, buffered to pH 4.5, over 20 min at imUmin; λ=254 nm; Deltapak® C18, 300 Å, 5 μm, 150×3.9 mm column); ¹H NMR (400 MHz, d₆-DMSO) 7.23 (1H, m), 7.28 (1H, m), 7.55 (2H, m), 7.99 (2H, dd, J 7.2 and 2.1 Hz), 8.30 (2H, dd, J 7.2 and 2.1 Hz) and 11.44 (1H, s).

### EXAMPLE 12

### 5-Chloro-1,3-benzoxazol-2-amine

Anhydrous copper (II) sulfate (1.1 to 10 equivalents, preferably 1.1 equivalents) and triethylamine (1.0 to 10 equivalents, preferably 1.0 equivalents) are added to a solution of N-(5-chloro-2-hydroxyphenyl)thiourea (1 equivalent) in an organic solvent, for example tetrahydrofuran, dichloromethane, or acetonitrile, and the mixture is stirred, between about 20° C. and 100° C., until the formation of the benzoxazole is complete. The reaction is filtered through a pad of diatomaceous earth, washed with solvent, and the combined filtrate is washed with 10% v/v aqueous ammonium hydroxide, dried over anhydrous magnesium sulfate and concentrated under reduced pressure to afford 5-chloro-1,3-benzoxazol-2-amine.

### EXAMPLE 13

### 2-{1-(2S,3R)-2-(2-Pyridyl)-3-(4-methoxyphenyl)pyrrolidinyl]-5-chlorobenzoxazole

Reaction of (2S,3R)-2-(2-pyridyl)-3-(4-methoxyphenyl)pyrrolidine (Yee et al., J. Org. Chem., 63(2), 326-330, (1998)) with 2-amino-4-chlorophenol (supplier: Aldrich, 1.0 equivalent), triethylamine (1 equivalent), carbon disulfide (1 equivalent), and hydrogen peroxide (30%, 1 equivalent) in tetrahydrofuran, under the conditions proposed by Li et al., J. Org. Chem., 62(13), 4539-4540, (1997), gives (2S,3R)-N1-(5-chloro-1,3-benzoxazol-2-yl)-3-(4-methoxyphenyl)-2-(2-pyridyl)-1-pyrrolidinecarbothioamide.

Anhydrous copper (II) sulfate (1.1 to 10 equivalents, preferably 1.1 equivalents) and triethylamine (1.0 to 10 equivalents, preferably 1.0 equivalents) is added to a solution of (2S,3R)-N1-(5-chloro-1,3-benzoxazol-2-yl)-3-(4-methoxyphenyl)-2-(2-pyridyl)-1-pyrrolidinecarbothioamide (1 equivalent) in an organic solvent, for example tetrahydrofuran, dichloromethane, or acetonitrile, and the mixture is stirred, between about 20° C. and 100° C., until the formation of the benzoxazole was complete. The reaction is filtered through a pad of diatomaceous earth, washed with solvent, and the combined filtrate is washed with 10% v/v aqueous ammonium hydroxide, dried over anhydrous magnesium sulfate and concentrated under reduced pressure to afford 2-{1-(2S,3R)-2-(2-pyridyl)-3-(4-methoxyphenyl)pyrrolidinyl]-5-chlorobenzoxazole.

### EXAMPLE 14

### N2-(Phenyl)-1,3-benzoxazol-2-amine using iron (III) chloride or manganese (III) acetate dihydrate

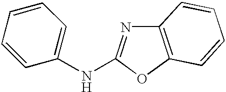

i). Preparation of N-(2-hydroxyphenyl)-N′-phenylthiourea

Phenyl isothiocyanate (800 μL, 0.90 g, 6.66 mmol) was added to a solution of 2-amino-phenol (0.727 g, 6.66 mmol) in tetrahydrofuran (THF) (90 mL) and the reaction was stirred at room temperature, under N₂, for 3 hours when the formation of the thiourea was complete (RP-HPLC Rt 6.394 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Triethylamine (930 μL, 6.66 mmol) was added and the reaction mixture was split into 9 equal portions (10 mL, 0.74 mmol (theoretical) of thiourea).

ii). Aminobenzoxazole formation using anhydrous iron (III) chloride

Anhydrous iron (II) chloride (0.132 g, 0.814 mmol, 1.1 eq.) was added to a solution of the thiourea reaction mixture (10 mL) and the reaction was stirred at ambient temperature for 48 h. The resulting suspension was filtered and the filtrate was concentrated to dryness. The residue was re-dissolved in dichloromethane (100 mL), filtered through a celite pad and washed with water (3×50 mL), and brine (50 mL). The organic layer was dried over anhydrous MgSO₄ and concentrated in vacuo to afford a brown solid (246 mg, 1.17 mmol, 135%). Further purification by dissolving the product in dichloromethane (10 mL), eluting through a diatomaceous earth pad (Varian ChemElut CE 1203, Extube®, pH 4.5) with dichloromethane (50 mL) and concentrating under reduced pressure afforded N2-(Phenyl)-1,3-benzoxazol-2-amine as a light brown solid (129 mg, 0.615 mmol, 71%); RP-HPLC Rt 7.999 min, 91% purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 7.03 (1H, t, J 7.2 Hz), 7.13 (1H, t, J 7.6 Hz), 7.22 (1H, t, J 7.6 Hz), 7.37 (2H, t, J 7.2 Hz), 7.46 (2H, m), 7.13 J 8.0 Hz), and 10.60 (1H, br s).

ii). Aminobenzoxazole formation using manganese (III) acetate dihydrate

Manganese (III) acetate dihydrate (0.218 g, 0.814 mmol, 1.1 eq.) was added to a solution of the thiourea reaction mixture (10 mL) and the reaction was stirred at ambient temperature for 48 h. The resulting suspension was filtered and the filtrate was concentrated to dryness. The residue was re-dissolved in dichloromethane (100 mL), filtered through a celite pad and washed with water (3×50 mL), and brine (50 mL). The organic layer was dried over anhydrous MgSO₄ and concentrated in vacuo to afford N2-(phenyl)-1,3-benzoxazol-2-amine as a red solid (359 mg, 230% of theory); RP-HPLC Rt 7.964 min, 83% organic purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 7.03 (1H, t, J 7.2 Hz), 7.13 (1H, t, J 7.6 Hz), 7.22 (1H, t, J 7.6 Hz), 7.37 (2H, t, J 7.2 Hz), 7.46 (2H, m), 7.76 (2H, d, J 8.0 Hz), and 10.60 (1H, br s).

### EXAMPLE 15

### N2-(4-Nitrophenyl)-1,3-benzoxazol-2-amine

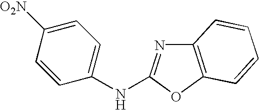

4-Nitrophenyl isothiocyanate (0.413 g, 2.29 mmol) was added to a solution of 2-amino-phenol (0.25 g, 2.29 mmol) in THF (15 mL) and the reaction was stirred at room temperature for 2 hours when the formation of the thiourea was complete (RP-HPLC Rt 7.174 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Anhydrous iron (III) chloride (0.409 g, 2.52 mmol) and triethylamine (0.30 mL, 2.29 mmol) were added, and the mixture was stirred at room temperature for 24 h. The resulting suspension was filtered through celite and the filtrate was concentrated to dryness. The residue was dissolved in dichloromethane (100 mL) and washed with saturated. aqueous CaCl2 (3×100 mL). The organic layer was dried over anhydrous MgSO₄ and concentrated in vacuo to afford a yellow-brown solid (575 mg,). Further purification by dissolving the product in dichloromethane (10 mL), eluting through a diatomaceous earth pad (Varian ChemElut CE 1203, Extube®, pH 4.5) with dichloromethane (50 mL) and concentrating under reduced pressure afforded N2-(4-Nitrophenyl)-1,3-benzoxazol-2-amine as a pink solid (569 mg, 97%); RP-HPLC Rt 8.364 min, 96% purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 7.21 (1H, t, J 1.2 Hz), 7.24 (1H, t, J 0.8 Hz), 7.55 (2H, m), 7.98 (2H, dd, J 9.2 and 2.8 Hz), 8.29 (2H, dd, J 9.2 and 2.8 Hz), and 11.44 (1H, br s).

### EXAMPLE 16

### N2-(4-Methoxyphenyl)-1,3-benzoxazol-2-amine

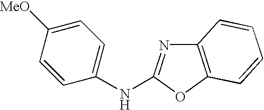

4-Methoxyphenyl isothiocyanate (0.321 mL, 2.29 mmol) was added to a solution of 2-amino-phenol (0.25 g, 2.29 mmol) in THF (15 mL) and the reaction was stirred at room temperature for 2 hours when the formation of the thiourea was complete (RP-HPLC Rt 6.388 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Anhydrous iron (III) chloride (0.409 g, 2.52 mmol) and triethylamine (0.30 mL, 2.29 mmol) were added, and the mixture was stirred at room temperature for 24 h. The resulting suspension was filtered through celite and the filtrate was concentrated to dryness. The residue was re-dissolved in dichloromethane (100 mL) and washed with sat. aq. CaCl₂ (3×100 mL). The organic layer was dried over anhydrous MgSO₄ and concentrated in vacuo to afford a brown solid (750 mg, 136%). Further purification by dissolving the product in dichloromethane (10 mL), eluting through a diatomaceous earth pad (Varian ChemElut CE 1203, Extube®, pH 4.5) with dichloromethane (50 mL) and concentrating under reduced pressure afforded N2-(4-Methoxyphenyl)-1,3-benzoxazol-2-amine as a light brown solid (546 mg, 99%); RP-HPLC Rt 7.808 min, 95% purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 3.36 (3H, s), 6.96 (2H, d, J 8.8 Hz), 7.09 (1H, t, J 7.6 Hz), 7.20 (1H, t, J 7.6 Hz), 7.41 (1H, d, J 8.0 Hz), 7.45 (1H, d, J 8.0 Hz), 7.67 (2H, d, J 8.8 Hz), and 10.44 (1H, br s).

### EXAMPLE 17

### N2-(3-pyridyl)-1,3-benzoxazol-2-amine

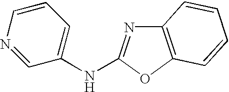

3-Pyridyl isothiocyanate (0.26 mL, 2.29 mmol) was added to a solution of 2-amino-phenol (0.25 g, 2.29 mmol) in THF (15 mL) and the reaction was stirred at room temperature for 2 hours when the formation of the thiourea was complete (RP-HPLC Rt 4.872 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Anhydrous iron (III) chloride (0.409 g, 2.52 mmol) and triethylamine (0.30 mL, 2.29 mmol) were added, and the mixture was stirred at room temperature for 24 h. The resulting suspension was filtered through celite and the filtrate was concentrated to dryness. The residue was re-dissolved in dichloromethane (100 mL) and washed with sat. aq. CaCl2 (3×100 mL). The organic layer was dried over anhydrous MgSO₄ and concentrated in vacuo to afford a gray solid (526 mg, 109%). Further purification by dissolving the product in dichloromethane (10 mL), eluting through a diatomaceous earth pad (Varian ChemElut CE 1203, Extube®), pH 4.5) with dichloromethane (50 mL) and concentrating under reduced pressure afforded N2-(3-pyridyl)-1,3-benzoxazo-2-lamine as a light gray solid (265 mg, 55%); RP-HPLC Rt 6.373 min, 95% purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 7.21 (1H, t, J 1.2 Hz), 7.24 (1H, t, J 0.8 Hz), 7.55 (2H, m), 7.98 (2H, dd, J 9.2 and 2.8 Hz), 8.29 (2H, dd, J9.2 and 2.8 Hz), and 11.44 (1H, br s).

### EXAMPLE 18

### Ethyl 3-(1,3-benzoxazol-2-ylamino)propanoate

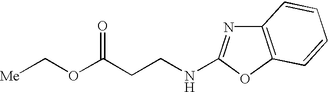

Ethyl 3-isothiocyanatopropionate (0.365 g, 2.29 mmol) was added to a solution of 2-amino-phenol (0.25 g, 2.29 mmol) in THF (15 mL) and the reaction was stirred at room temperature for 2 hours when the formation of the thiourea was complete (RP-HPLC Rt 5.957 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Anhydrous iron (III) chloride (0.409 g, 2.52 mmol) and triethylamine (0.30 mL, 2.29 mmol) were added, and the mixture was stirred at room temperature for 1 h. The resulting suspension was filtered through celite and the filtrate was concentrated to dryness. The residue was re-dissolved in dichloromethane (100 mL) and washed with saturated aqueous CaCl₂ (3×100 mL). The organic layer was dried over anhydrous MgSO₄ and concentrated in vacuo to afford a brown solid (590 mg, 110%). Further purification by dissolving the product in dichloromethane (10 mL), eluting through a diatomaceous earth pad (Varian ChemElut CE 1203, Extube®, pH 4.5) with dichloromethane (50 mL) and concentrating under reduced pressure afforded ethyl 3-(1,3-benzoxazol-2-ylamino)propanoate as a pink solid (203 mg, 38%); RP-HPLC Rt 6.601 min, 80% purity (5% to 95% acetonitrile/0.1M aqueous ammonium acetate, buffered to pH 4.5, over 10 min at 1 ml/min; λ=254 nm; Waters Deltapak® C18, 300 Å, 5 μm, 150×3.9 mm column); and m/z 235 (MH⁺).

### EXAMPLE 19

### N2-(4-Methyl-2-Nitrophenyl)-1,3-benzoxazol-2-amine

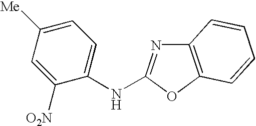

4-Methy-2-Nitrophenyl isothiocyanate (0.445 g, 2.29 mmol) was added to a solution of 2-amino-phenol (0.25 g, 2.29 mmol) in THF (15 mL) and the reaction was stirred at room temperature for 2 hours when the formation of the thiourea was complete (RP-HPLC Rt 7.388 min, (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column). Anhydrous iron (III) chloride (0.409 g, 2.52 mmol) and triethylamine (0.30 mL, 2.29 mmol) were added, and the mixture was stirred at room temperature for 24 h. The resulting suspension was filtered through celite and the filtrate was concentrated to dryness. The residue was re-dissolved in dichloromethane (100 mL) and washed with saturated aqueous CaCl₂ (3×100 mL). The organic layer was dried over anhydrous MgSO₄ and concentrated in vacuo. Further purification by dissolving the product in dichloromethane (10 mL), eluting through a diatomaceous earth pad (Varian ChemElut CE 1203, Extube®, pH 4.5) with dichloromethane (50 mL) and concentrating under reduced pressure afforded N2-(4-methyl-2-nitrophenyl)-1,3-benzoxazol-2-amine as a yellow solid (574 mg, 93%); RP-HPLC Rt 9.688 min, 89% purity (5% to 95% acetonitrile/0.1M aqueous ammonium acetate, buffered to pH 4.5, over 10 min at 1 mL/min; λ=254 nm; Waters Deltapak® C18, 300 Å, 5 μm, 150×3.9 mm column); and ¹H NMR (400 MHz, d₆-DMSO) 2.39 (3H, s,), 7.17 (1H, t, J 7.6 Hz), 7.25 (1H, t, J 7.6 Hz), 7.44 (1H, br d, J 6.0 Hz), 7.51 (1H, d, J 8.0 Hz), 7.63 (1H, d, J8.4 Hz), 7.94 (1H, s), 8.18 (1H, br s), and 10.50 (1H, br s).

What is claimed is:

1. A method of making an optionally substituted 2-amino-benzoxazole or 2-amino-benzimidazole which comprises reacting a corresponding optionally substituted N-(2-hydroxyphenyl)thiourea or N-(2-aminophenyl)thiourea, respectively, with a transition metal in its I or II oxidation state, in the presence or absence of a base to obtain the optionally substituted 2-amino-benzoxazole or 2-aminobenzimidazole.

2. A process for the synthesis of a compound of formula (II), 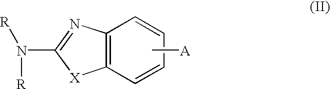
comprising the step of reacting a compound of formula (I), 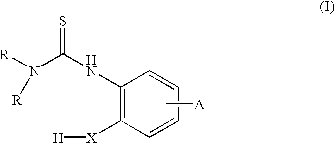
with a transition metal in its I or II oxidation state and optionally a base until the reaction is substantially complete to obtain the compound of formula (II); wherein:
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R³, —W—(CH₂), —NR^{d}R^{e}, —W—(CH₂)ₜ—O-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

3. A process for the synthesis of a compound of formula (II), 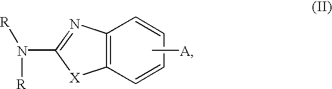
comprising the step of reacting an isothiocyanate, an optionally substituted 2-(X)-aniline, a transition metal in its I or II oxidation state and optionally a base, until the reaction is substantially complete to obtain a compound of formula (II) wherein
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and
X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

4. A process according to claim 3 wherein the isothiocyanate is of the formula R-NCS and the optionally substituted aniline is of the formula

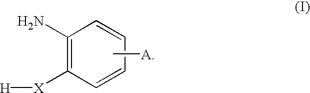

5. A process for the synthesis of a compound of formula (II), 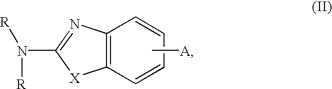
comprising the steps:
forming an isothiocyanate in situ by reacting an amine or an aniline with a reagent having a thiocarbonyl moiety and which is capable of a double nucleophilic attack at the carbon of the thiocarbonyl moiety to yield the isothiocyanate;
reacting the isothiocyanate with an optionally substituted 2-(X)-aniline, a transition metal in its I or II oxidation state and optionally a base, until the reaction is substantially complete to obtain a compound of formula (II), wherein
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R^{e}, —W—(CH₂)ₜNR^{d}R^{e}, —W—(CH₂)ₜO-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and
X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

6. The process according to claim 1, 2, 4 or 5, wherein the transition metal is Cr, Mn, Fe, Co, Cu or Zn, or a combination thereof and wherein the base is selected from the group consisting of triethylamine, ammonia, sodium hydroxide, sodium hydrogen carbonate and cesium carbonate.

7. The process according to claim 6, wherein the transition metal is a corresponding salt or a combination of salts.

8. The process according to claim 6, wherein the transition metal is selected from the group consisting of copper (II) sulfate, anhydrous copper (II) sulfate or copper (I) chloride.

9. A method of making an optionally substituted 2-amino-benzoxazole or 2-amino-benzimidazole which comprises reacting a corresponding optionally substituted N-(2-hydroxyphenyl)thiourea or N-(2-aminophenyl)thiourea, respectively, with a transition metal in its III oxidation state, in the presence or absence of a base to obtain the optionally substituted 2-amino-benzoxazole or 2-aminobenzimidazole.

10. A process for the synthesis of a compound of formula (II), 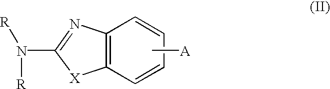
comprising the step of reacting a compound of formula (I), 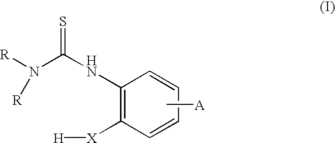
with a transition metal in its III oxidation state and optionally a base until the reaction is substantially complete to obtain the compound of formula (II); wherein:
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z₂, —Z¹—N(R¹)—S(O)₂—Z₂, —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

11. A process for the synthesis of a compound of formula (II), 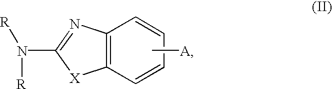
comprising the step of reacting an isothiocyanate, an optionally substituted 2-(X)-aniline, a transition metal in its III oxidation state and optionally a base, until the reaction is substantially complete to obtain a compound of formula (II) wherein
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z², —Z¹—N(R¹)—S(O)₂—Z², —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and
X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

12. A process according to claim 11 wherein the isothiocyanate is of the formula R-NCS and the optionally substituted aniline is of the formula

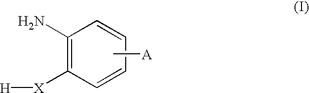

13. A process for the synthesis of a compound of formula (II), 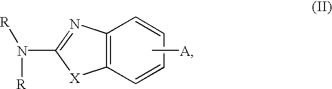
comprising the steps:
forming an isothiocyanate in situ by reacting an amine or an aniline with a reagent having a thiocarbonyl moiety and which is capable of a double nucleophilic attack at the carbon of the thiocarbonyl moiety to yield the isothiocyanate;
reacting the isothiocyanate with an optionally substituted 2-(X)-aniline, a transition metal in its III oxidation state and optionally a base, until the reaction is substantially complete to obtain a compound of formula (II), wherein
A represents one or more substituents, each independently selected from the group consisting of hydrogen, halogen, —CN, —NO₂, —C(O)OH, —C(O)H, and —OH, or is an optionally substituted moiety each independently selected from the group consisting of —C(O)O-alkyl, —C(O)O-aryl, —C(O)O-heterocyclyl, —C(O)-alkyl, —C(O)-aryl, —C(O)-heterocyclyl, carboxamido, tetrazolyl, trifluoromethylcarbonylamino, trifluoromethylsulfonamido, alkyl, cycloalkyl, alkoxy, aryl, heterocyclyl, alkenyl, alkynyl, aryloxy, heterocyclyloxy, heterocyclylalkoxy, arylalkoxy, alkyl-S(O)ₚ—, alkyl-S—, aryl-S, heterocyclyl-S—, aryl-S(O)ₚ—, heterocyclyl-S(O)ₚ—, arylalkyl, heterocyclylalkyl, cycloalkylalkyl, amino, aminoalkyl, amido, —Z¹—C(O)N(R¹)₂, —Z¹—N(R¹)—C(O)—Z₂, —Z¹—N(R¹)—S(O)₂—Z₂, —Z¹—N(R¹)—C(O)—N(R¹)—Z², and CH₂OR²;
where R¹ for each occurrence is independently H, or optionally substituted alkyl, heterocyclyl, aryl, aralkyl or heterocyclylalkyl;
p is 1 or 2;
R² for each occurrence is independently hydrogen, or optionally substituted alkyl, aryl, heterocyclyl, —CH₂—NR^{d}R^{e}, —W—(CH₂)ₜ—NR^{d}R^{e}, —W—(CH₂)ₜ—O-alkyl, —W—(CH₂)ₜ—S-alkyl, or —W—(CH₂)ₜ—OH;
R^{d} and R^{e} for each occurrence are independently H, alkyl, alkanoyl or SO₂-alkyl; or R^{d}, R^{e} and the nitrogen atom to which they are attached together form a five- or six-membered heterocyclic ring;
W is a covalent bond, O, S, S(O), S(O)₂ or NR^{f}, where R^{f} is H or alkyl;
t for each occurrence is independently an integer from 2 to 6;
Z¹ is a covalent bond or alkyl;
Z² is an optionally substituted alkyl, aryl, heterocyclyl, arylalkyl, or heterocyclylalkyl;
R for each occurrence is independently hydrogen or silyl or is independently an optionally substituted moiety selected from the group consisting of alkyl, arylalkyl, heterocyclylalkyl, aryl, heterocyclyl, cycloalkyl, and cycloalkylalkyl; or each R is taken together with the nitrogen atom to which they are attached to form an optionally substituted 5- or 6-membered ring optionally having one or more other heteroatoms selected from the group consisting of N, O and S; and
X is O, NH, N-alkyl, N-cycloalkyl, N-arylalkyl, N-heterocyclylalkyl, N-sulfonyl, N-carboxyl, N-aryl, or N-heterocyclyl wherein the group attached to the nitrogen is optionally substituted with one or more substituents.

14. The process according to claim 9, 10, 12, or 13, wherein the transition metal is Cr, Mn, Fe, or Co or a combination thereof and wherein the base is selected from the group consisting of triethylamine, ammonia, sodium hydroxide, sodium hydrogen carbonate and cesium carbonate.

15. The process according to claim 14, wherein the transition metal is a corresponding salt or a combination of salts.

16. The process according to claim 14, wherein the transition metal salt is selected from the group consisting of iron (III) chloride, anhydrous iron (III) chloride, manganese (III) acetate dihydrate and anhydrous manganese (III) diacetate.
